(19) United States
(12) Patent Application Publication
(10) Pub. No.: US 2010/0167200 A1
(43) Pub. Date: Jul. 1, 2010

(54) (Meth)acrylate compound, photosensitive polymer, and resist composition including the same

(76) Inventors: Sang-Jun Choi, Uiwang-si (KR); Sung-Jae Lee, Uiwang-si (KR); Seung-Jib Choi, Uiwang-si (KR)

Correspondence Address: LEE & MORSE, P.C., 3141 FAIRVIEW PARK DRIVE, SUITE 500, FALLS CHURCH, VA, 22042, US

(21) Appl. No.: 12/654,740

(22) Filed: Dec. 30, 2009

(30) Foreign Application Priority Data
Dec. 31, 2008 (KR) 10-2008-0138787

(51) Int. Cl. G03F 7/004; C07D 307/12; C07C 69/608; C08F 24/00; C08F 32/08

(52) U.S. Cl. 430/270.1; 549/323; 560/117; 526/270; 526/281

(57) ABSTRACT

A (meth)acrylate compound having an acid-labile ester group, a photosensitive polymer, and a resist composition including the same, the (meth)acrylate compound being represented by the following Chemical Formula 1

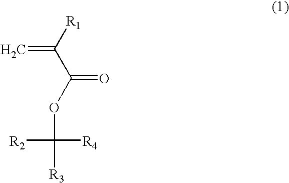

wherein, R₁ is hydrogen or methyl, R₂ and R₃ are each independently a substituted or unsubstituted linear alkyl, a substituted or unsubstituted cyclic alkyl, or linked each other to form a monocyclic ring or a fused-ring, and R₄ is a linear ester or cyclic ester group.

### BACKGROUND

1. Field of the Invention

Embodiments relate to a (meth)acrylate compound, a photosensitive polymer, and a resist composition including the same.

2. Description of the Related Art

Recently, semiconductor manufacturing processes and integration of semiconductors have increasingly included forming a fine pattern. A photoresist material using a shorter wavelength, e.g., an ArF excimer laser of 193 nm, has been preferred to one using a conventional KrF excimer laser of 248 nm.

However, since a semiconductor device with a capacity of more than 16 gigabytes may need a pattern size of less than 70 nm according to a design rule, a thickness of a resist film may become thinner. Furthermore, a process margin for underlayer etching has been reduced, and thus a resist material using an ArF excimer laser also may have reached a limit.

### SUMMARY

Embodiments are directed to a (meth)acrylate compound, a photosensitive polymer, and a resist composition including the same.

It is a feature of an embodiment to provide a (meth)acrylate compound including an acid-labile ester group that may be used to prepare a polymer at a low cost, which exhibits excellent resistance to dry etching and adherence to an underlayer.

It is another feature of an embodiment to provide a resist composition, e.g., a chemical-amplification type resist composition, including the photosensitive polymer and providing excellent lithography performance in a lithographic process using an ultrashort wavelength region, e.g., a 193 nm region and EUV (13.5 nm) as a light source.

At least one of the above and other features and advantages may be realized by providing a (meth)acrylate compound having an acid-labile ester group, the (meth)acrylate compound being represented by the following Chemical Formula 1

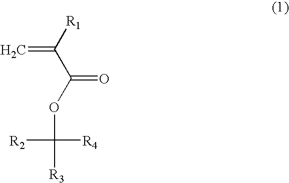

wherein, R₁ is hydrogen or methyl, R₂ and R₃ are each independently a substituted or unsubstituted linear alkyl, a substituted or unsubstituted cyclic alkyl, or linked to each other to form a cyclic ring or a fused-ring, and R₄ is a linear ester or cyclic ester group.

R₂ and R₃ may be the cyclic ring linking R₂ and R₃, the cyclic ring including cyclopentyl, cyclohexyl, adamantyl, or isobornyl.

R₄ may be γ-butyrolactonyl or —(CH₂)ₙCOOR′, n being an integer of 1 to 3 and R′ being a C1 to C3 alkyl.

The (meth)acrylate compound may include at least one compound selected from the group consisting of compounds represented by the following Chemical Formulae 1a to 1j:

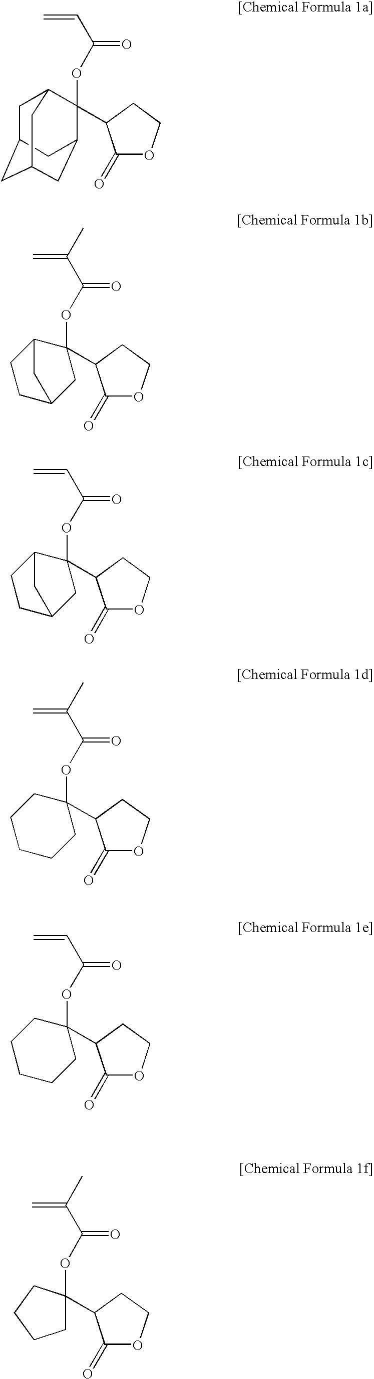

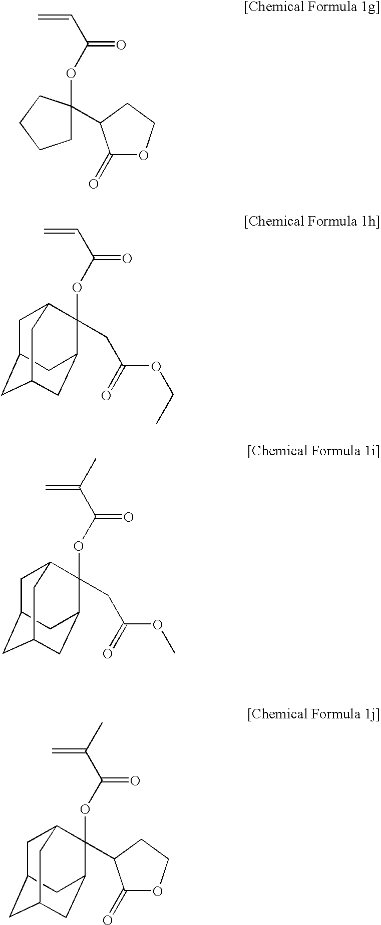

At least one of the above and other features and advantages may also be realized by providing a photosensitive polymer including a repeating unit derived from a compound represented by the following Chemical Formula 1:

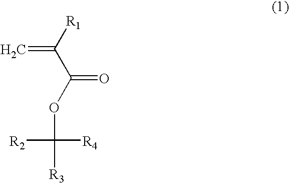

wherein, R₁ is hydrogen or methyl, R₂ and R₃ are each independently a substituted or unsubstituted linear alkyl, a substituted or unsubstituted cyclic alkyl, or linked each other to form a monocyclic ring or a fused-ring, and R₄ is a linear ester or cyclic ester group.

The photosensitive polymer may have a weight average molecular weight (Mw) of about 3,000 to about 20,000.

The photosensitive polymer may have a polydispersity (Mw/Mn) of about 1.3 to about 2.5.

The photosensitive polymer may further include at least one repeating unit derived from a compound represented by the following Chemical Formula 2:

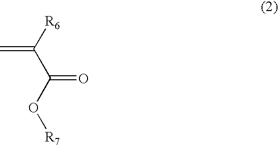

wherein, in Chemical Formula 2, R₆ is hydrogen or methyl, and R₇ is hydrogen, a bulky alkyl, an alkyl including a polar functional group, or a cycloalkyl including a polar functional group.

R₇ may be the bulky alkyl, the bulky alkyl being unsubstituted norbornyl, norbornyl substituted with a lower alkyl, unsubstituted isobornyl, isobornyl substituted with a lower alkyl, unsubstituted cyclodecanyl, cyclodecanyl substituted with a lower alkyl, unsubstituted adamantyl, adamantyl substituted with a lower alkyl, alkoxycarbonyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, a tertiary alkyl, or acetal.

R₇ may be 2-hydroxyethyl or 3-hydroxy-1-adamantyl.

A mole ratio of repeating units derived from compounds represented by Chemical Formula 1 to all repeating units derived from compounds represented by Chemical Formula 2 is about 3:7 to about 7:3.

At least one of the above and other features and advantages may also be realized by providing a resist composition including the photosensitive polymer as claimed in claim 5, a photoacid generator, and an organic solvent.

The photosensitive polymer may be included in an amount of about 5 to about 15 parts by weight, based on 100 parts by weight of the resist composition.

The photoacid generator may be included in an amount of about 1 to about 15 parts by weight, based on 100 parts by weight of the photosensitive polymer.

The photoacid generator may include at least one of triarylsulfonium salts, diaryliodonium salts, and sulfonates.

The composition may further include about 0.1 to about 1.0 part by weight of an organic base, based on 100 parts by weight of the photosensitive polymer.

The organic base may include at least one of triethylamine, triisobutylamine, trioctylamine, triisodecylamine, and triethanolamine.

### DETAILED DESCRIPTION

Korean Patent Application No. 10-2008-0138787, filed on Dec. 31, 2008, in the Korean Intellectual Property Office, and entitled: “(Meth)Acrylate Compound and Photosensitive Polymer, and Resist Composition,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another element, it can be directly on the other element, or intervening elements may also be present. Further, it will be understood that when an element is referred to as being “under” another element, it can be directly under, and one or more intervening elements may also be present. Like reference numerals refer to like elements throughout.

Exemplary embodiments will hereinafter be described in detail. However, these embodiments are only exemplary but do not limit the scope.

As used herein, when specific definition is not otherwise provided, the term “alkyl” refers to a C2 to C14 alkyl, the term “lower alkyl” refers to a C1 to C4 alkyl, the term “alkoxy” refers to a C1 to C20 alkoxy, for example a C1 to C12 alkoxy, the “cyclic ring” refers to a C2 to C14 cyclic ring, for example a cycloalkyl, and the term “ester” refers to a C3 to C10 ester.

In the present specification, the term “substituted” refers to a functional group substituted with an alkyl or an aryl instead of at least one of hydrogen.

In an embodiment, a (meth)acrylate compound including an acid-labile ester group may be represented by the following Chemical Formula 1.

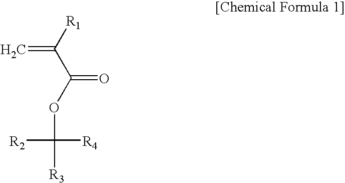

In Chemical Formula 1, R₁ may be, e.g., hydrogen or methyl. In Chemical Formula 1, R₂ and R₃ may each independently be, e.g., a linear or cyclic, substituted or unsubstituted, alkyl, or may be linked each other to form a monocyclic ring or a fused-ring. In an implementation, R₂ and R₃ may each independently be, e.g., methyl, ethyl, cyclopentyl, cyclohexyl, adamantyl, isobornyl, or a substituent thereof. In another implementation, R₂ and R₃ may be linked to form a cyclic ring, e.g., cyclopentyl, cyclohexyl, adamantyl, isobornyl, and the like.

In Chemical Formula 1, R₄ may be, e.g., a linear ester or cyclic ester group. In an implementation, R₄ may be, e.g., γ-butyrolactonyl or —(CH₂)ₙCOOR′, where n is an integer of about 1 to about 3, and R′ is a C1 to C3 alkyl.

Specific examples of the (meth)acrylate compound represented by Chemical Formula 1 are represented by Chemical Formulae 1a to 1j:

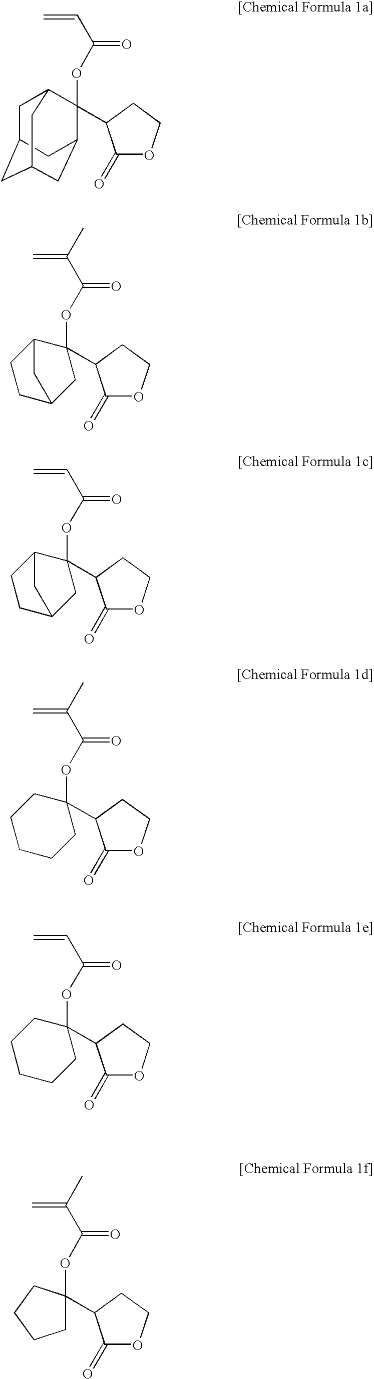

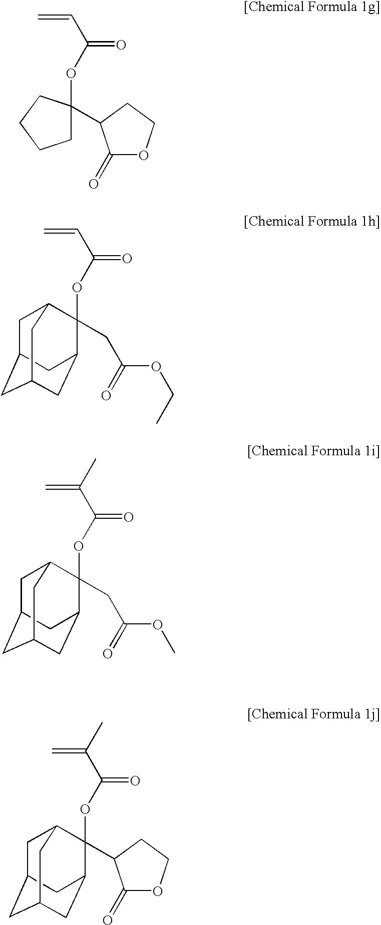

The (meth)acrylate compound represented by Chemical Formula 1 may include one or more compounds represented by the Chemical Formulae 1a to 1j, but are not limited thereto

The (meth)acrylate compound represented by Chemical Formula 1 may be decomposed under an acid catalyst.

The (meth)acrylate compound represented by Chemical Formula 1 may be prepared through the following process.

First, an α-haloester and a Zn agent may be reacted. The resultant product may react with a ketone compound through a Reformatsky reaction to prepare a β-hydroxy-ester.

The α-haloester may include, e.g., a bromine ester such as α-bromo-γ-butyrolactone and/or ethylbromo acetate. The ketone compound may include, e.g., 2-adamantanone, cyclohexanone, and the like. The α-haloester and the ketone compound may be selected based on a desired substituent of R₂, R₃, and R₄ of the above Chemical Formula 1 in the final product.

A mole ratio of the α-haloester to the ketone compound may also be selected according to the desired final product. The reaction of the α-haloester and Zn agent may include a catalyst, e.g., CuCl, and/or a solvent, e.g., tetrahydrofuran.

The prepared β-hydroxy-ester compound may react with acryloyl halide (i.e., acroyl halide), e.g., acryloyl chloride, or methacryloyl halide (i.e., methacroyl halide), e.g., methacryloyl chloride, under a nitrogen-containing compound to prepare the (meth)acrylate compound represented by Chemical Formula 1. The nitrogen-containing compound may include, e.g., triethylamine, pyridine, and the like. The amount of the β-hydroxy-ester compound, nitrogen-containing compound, and acryloyl halide or methacryloyl halide may be controlled according to the desired final product.

An embodiment provides a photosensitive polymer including the (meth)acrylate compound represented by Chemical Formula 1. In other words, the photosensitive polymer may include repeating units derived from the (meth)acrylate compound represented by Chemical Formula 1.

The photosensitive polymer may be a homopolymer formed by polymerizing the (meth)acrylate monomer of the above Chemical Formula 1. In an implementation, the photosensitive polymer may be a copolymer including a repeating unit derived from the (meth)acrylate compound represented by Chemical Formula 1 and at least one repeating unit derived from a compound represented Chemical Formula 2, below. The photosensitive polymer may be any suitable type of copolymer, e.g., a block copolymer including regularly repeated repeating units derived from the compounds represented by Chemical Formula 1 and Chemical Formula 2, or a random copolymer including randomly repeated repeating units. In another implementation, the photosensitive polymer may have three different repeating units, and may be a terpolymer. In still another implementation, the photosensitive polymer may have four different repeating units.

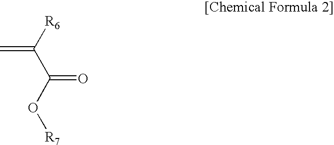

In Chemical Formula 2, R₆ may be, e.g., hydrogen or methyl. In Chemical Formula 2, R₇ may be hydrogen, a bulky alkyl, an alkyl including a polar functional group, or a cycloalkyl including a polar functional group.

The bulky alkyl of the above R₇ may include, e.g., unsubstituted norbornyl, norbornyl substituted with a lower alkyl, unsubstituted isobornyl, isobornyl substituted with a lower alkyl, unsubstituted cyclodecanyl, cyclodecanyl substituted with a lower alkyl, unsubstituted adamantyl, adamantyl substituted with a lower alkyl, alkoxycarbonyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, a tertiary alkyl, or acetal. In an implementation, the bulky alkyl may include, e.g., 1-methyl-1-cyclohexyl, 2-methyl-2-adamantyl, 2-ethyl-2-adamantyl, 2-hydroxyethyl, 3-hydroxy-1-adamantyl, 2-methyl-2-norbornyl, 2-ethyl-2-norbornyl, 2-methyl-2-isobornyl, 2-ethyl-2-isobornyl, 8-methyl-8-tricyclodecanyl, 8-ethyl-8-tricyclodecanyl, 2-propyl-2-adamantyl, t-butoxycarbonyl, t-butoxycarbonylmethyl, t-amyloxycarbonyl, t-amyloxycarbonylmethyl, 1-ethoxyethoxycarbonylmethyl, 2-tetrahydropyranyloxycarbonylalkyl, 2-tetrahydrofuranyloxycarbonylalkyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, triethylcarbamyl, 1-ethylcyclopentyl, or t-amyl.

The alkyl-group or cycloalkyl group including a polar functional group of R₇ may include, e.g., 2-hydroxyethyl or 3-hydroxy-1-adamantyl.

When the photosensitive polymer is a copolymer further including a repeating unit derived from the compound represented by Chemical Formula 2, a mole ratio of repeating units derived from the compounds represented by Chemical Formulae 1 and 2 may be, e.g., about 3:7 to about 7:3. In other words, a mole ratio of repeating units derived from the compound represented by Chemical Formula 1 to all repeating units derived from compounds represented by Chemical Formula 2 may be, e.g., about 3:7 to about 7:3.

The photosensitive polymer may have a weight average molecular weight (Mw) of about 3,000 to about 20,000. Maintaining the Mw of the photosensitive polymer at about 3,000 to about 20,000 may help ensure that a resultant resist exhibits excellent, i.e., reduced, line edge roughness (LER). In an implementation, the Mw may be about 5,000 to about 10,000.

The photosensitive polymer may have a polydispersity (Mw/Mn) of about 1.3 to about 2.5. Maintaining the polydispersity at about 1.3 to about 2.5 may help ensure that a resultant resist has both an excellent etching resistance and a resolution. In an implementation, the polydispersity may be about 1.5 to about 2.0.

The photosensitive polymer according to an embodiment may be a polymer obtained from compounds including, e.g., a functional acid-labile ester group. The photosensitive polymer may be advantageously used to prepare a resist composition having excellent dry etching resistance. The (meth)acrylate compound including an acid-labile ester group, e.g., a monomer starting material for a repeating unit, may be easily decomposed under an acid catalyst. Therefore, a photosensitive polymer obtained from these compounds may have an improved etching resistance compared with a polymer having, e.g., an acid-labile adamantyl group. The photosensitive polymer of an embodiment may exhibit excellent dry etching resistance compared to other ArF resist materials. Thus, the photosensitive polymer of an embodiment may be used to form an etching mask for a semiconductor device in which a higher resolution is desired. In other words, when a resist composition including the photosensitive polymer is used in a photolithography process, it may provide excellent lithography performance.

Another embodiment may provide a resist composition including the photosensitive polymer.

The resist composition may include, e.g., the photosensitive polymer, a photoacid generator (PAG), and a solvent.

Hereinafter, components of the resist composition according to an embodiment are described in more detail.

Photosensitive Polymer

The photosensitive polymer may be the same photosensitive polymer of an embodiment, as described above.

The photosensitive polymer may be included in an amount of about 5 to about 15 parts by weight, based on 100 parts by weight of the resist composition. Maintaining the amount of the photosensitive polymer at about 5 to about 15 parts by weight may help ensure excellent etching resistance and adhesion characteristics in the resist composition and a resultant resist.

Photoacid Generator (PAG)

The photoacid generator may include, e.g., an inorganic onium salt, organic sulfonate, and/or mixtures thereof. In an implementation, the photoacid generator may include, e.g., sulfonate or iodonium salt such as a triarylsulfonium salt, a diaryl iodonium salt, sulfonate, and/or mixtures thereof. In an implementation, the photoacid generator may include, e.g., triarylsulfonium triflate, diaryliodonium triflate, triarylsulfonium nonaflate, diaryliodonium nonaflate, succinimidyl triflate, 2,6-dinitrobenzyl sulfonate, and/or mixtures thereof.

The photoacid generator may be included in an amount of about 1 to about 15 parts by weight, based on 100 parts by weight of the copolymer. Maintaining the amount of the photoacid at about 1 to about 15 parts by weight may help ensure that an exposure dose with respect to the resist composition and transmission of the resist composition may be appropriately controlled.

Solvent

The solvent may include, e.g., propylene glycol monomethyl ether acetate (PGMEA), propylene glycol methyl ether (PGME), ethyl lactate (EL), cyclohexanone, 2-heptanone, and the like.

The solvent may be included as the balance amount of the resist composition without limitation. In an implementation, the solvent may be included in amount of about 80 parts by weight to about 95 parts by weight, based on 100 parts by weight of the resist composition.

Additive

The resist composition may further include an additive. The additive may include, e.g., an organic base as a quencher in order to control the exposure dose and to form a resist profile.

The organic base may include, e.g., an amine-based compound such as triethylamine, triisobutylamine, trioctylamine, triisodecylamine, triethanolamine, and/or mixtures thereof.

In an implementation, the organic base may be included in an amount of about 0.1 to about 1 part by weight, based on 100 parts by weight of the polymer. Maintaining the amount of the organic base at about 0.1 to about 1 part by weight may help ensure that the exposure dose is not excessively increased, a desirable effect may be obtained, and the pattern is well formed.

A process to form a desirable pattern with the resist composition of an embodiment may be as follows.

A bare silicon wafer or a silicon wafer including an underlayer, e.g., a silicon oxide layer, a silicon nitride layer, or a silicon nitride oxide layer, on an upper surface thereof may be treated with, e.g., HMDS (hexamethyldisilazane) or an organic anti-reflection coating (bottom anti-reflective coating). Then, the resist composition may be coated on the silicon wafer at a thickness of about 100 to about 150 nm to provide a resist layer.

The silicon wafer with the resist layer thereon may be soft-baked (SB), i.e., pre-baked, at a temperature of about 90 to about 120° C. for about 60 to about 90 seconds to remove the solvent. The soft-baked wafer may then be exposed to a variety of exposure light sources, e.g., ArF or EUV (extreme UV), E-beam, and so on. In order to perform chemical reaction in the exposure region of the resist layer, i.e., to make the exposed portion more soluble in a developer solution, the wafer may be subjected to a PEB (post-exposure baking) at a temperature of about 90 to about 120° C. for about 60 to about 90 seconds.

Then, the resist layer may be developed in a basic aqueous developing solution. The exposure region may have very high solubility in the basic aqueous developing solution, so it may be easily dissolved and removed during the development. In an impelementation, e.g., tetramethylammonium hydroxide (TMAH) may be used as the basic aqueous developing solution. When the exposure light source is an ArF excimer laser, a 70 to 100 nm line end space pattern may be obtained at a dose of about 20 to about 50 mJ/cm².

The resist pattern obtained from the above process may be used as a mask, and the underlayer, e.g., a silicon oxide layer, may be etched using an etching gas, e.g., a plasma of halogen gas or fluorocarbon gas such as CF₄. Portions of the resist pattern that remains on the wafer may be removed by using a stripper to provide a desired silicon oxide layer pattern.

The following examples are suggested for helping in understanding of the embodiments, but the embodiments are not limited to the following examples.

### Example 1-1

### Synthesis of Monomer (I)

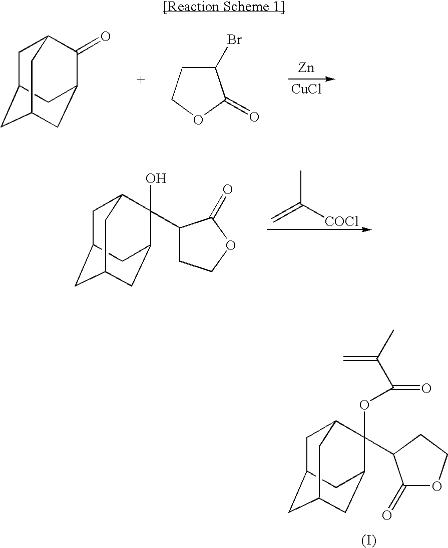

According to the method as shown in Reaction Scheme 1, a monomer (I) was synthesized.

0.11 mol of α-bromo-γ-butyrolactone was dissolved in tetrahydrofuran. Then, the solution was reacted under 0.17 mol of Zn and a CuCl catalyst at 45° C. for 2 hours.

Then, 0.1 mol of 2-adamantanone was added into the reaction product at room temperature, and the mixture was reacted according to a Reformatsky reaction for 8 hours. The obtained reactant was added dropwise into water, treated with diluted sulfuric acid, and extracted using diethyl ether. The extracted product was purified with a column chromatography (hexane:ethyl acetate=2:1 volume ratio), and the purified product was recrystallized (yield: 80%).

0.1 mol of the obtained product and 0.11 mol of triethylamine were dissolved in tetrahydrofuran. 0.1 mol of methacryloyl chloride was slowly added dropwise into the obtained solution in an ice bath, and then reacted at about 45° C. for 4 hours. When the reaction was complete, the reactant was slowly neutralized in an excess of a diluted hydrochloric acid solution, and then, extracted using ethyl ether. The extracted product was purified with column chromatography (hexane:ethyl acetate=2:1 volume ratio) (yield: 70%) to prepare a monomer (I).

NMR of the prepared monomer (I) was as follows.

¹H-NMR (CDCl₃, ppm):

6.4-6.5 (dd, 2H, vinyl), 4.3 (m, 2H, —CO₂CH₂),

2.9 (t, 1H, —CHCO₂), 2.0 (s, 3H, —CH₃),

2.2 (m, 2H, —CH₂—), 1.9-2.1 (m, 16H, —CH₂—, —CH—)

### Example 1-2

### Synthesis of Monomer (II)

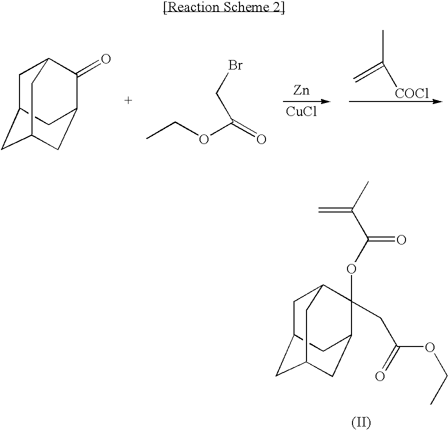

According to the method as shown in Reaction Scheme 1, a monomer (II) was synthesized.

The same process as in Example 1-1 was performed, except that ethylbromoacetate was used instead of α-bromo-γ-butyrolactone to prepare a monomer (II) (yield: 60%).

NMR of the prepared monomer (II) was as follows.

¹H-NMR (CDCl₃, ppm):

6.4-6.5 (dd, 2H, vinyl), 4.2 (q, 2H, —CO₂CH₂),

2.5 (s, 2H, —CH₂CO₂), 2.0 (s, 3H, —CH₃),

2.2 (m, 2H, —CH₂—), 1.2-1.8 (m, 17H, —CH₂—, —CH—)

### Example 1-3

### Synthesis of Monomer (III)

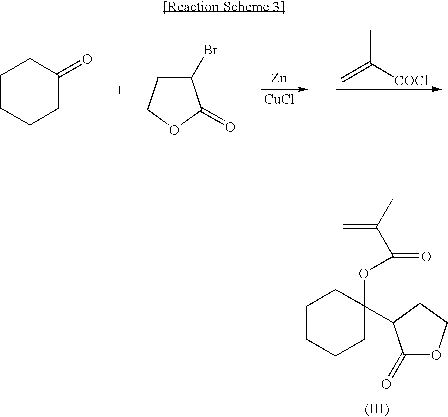

According to the method as shown in Reaction Scheme 3, a monomer (III) was synthesized.

The same process as in Example 1-1 was performed, except that cyclohexanone was used instead of 2-adamantanone to prepare a monomer (III) (yield: 60%).

### Example 2-1

### Synthesis of Photosensitive Polymer

40 mmol of the monomer (I) synthesized according to Example 1-1, 30 mmol of 1-adamantyl methacrylate (AMA), and 30 mmol of 3-hydroxy-1-adamantyl methacrylate (HAMA) were put in a round flask. In the round flask, propylene glycol monomethyl ether acetate (PEGMA) solvent was added in a 2:1 weight ratio of solvent to the total weight of monomers therein to dissolve the monomers. Then, 15 mmol of 2′-azobis(2-methylpropinonate) (V601, Wako Pure Chemical Industries Ltd.) was added thereto as a polymerization initiator. The mixture solution was polymerized at a temperature of 80° C. for 4 hours.

When the polymerization was complete, the reactant was slowly precipitated in an excess of an isopropyl alcohol solvent, and then, the precipitate was filtered. The filtered precipitate was dissolved in a predetermined amount of tetrahydrofuran, and the solution was re-precipitated in an isopropyl alcohol solvent. Then, the precipitate was dried in a 50° C. vacuum oven for 24 hours, obtaining a photosensitive polymer with repeating units represented by the following Chemical Formulae 3a, 3b, and 3c (yield: 55%). The photosensitive polymer had a weight average molecular weight (Mw) of 7,700 and polydispersity (Mw/Mn) of 1.6. The mole ratio (l:m:n) of the repeating units represented by Chemical Formulae 3a, 3b, and 3c was 4:3:3.

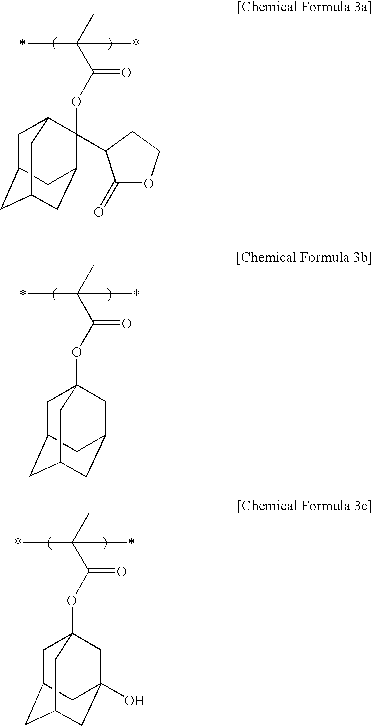

### Example 2-2

### Synthesis of Photosensitive Polymer

40 mmol of the monomer (II) synthesized according to Example 1-2, 30 mmol of 1-adamantyl methacrylate (AMA), and 30 mmol of 3-hydroxy-1-adamantyl methacrylate (HAMA) were put in a round flask, and then, polymerized according to the same method as in Example 2-1, thus obtaining a photosensitive polymer including repeating units represented by the following Chemical Formulae 4a, 3b, and 3c (yield: 60%). The photosensitive polymer had a weight average molecular weight (Mw) of 7,500 and polydispersity (Mw/Mn) of 1.7. The mole ratio (l:m:n) of the repeating units represented by Chemical Formulae 4a, 3b and 3c was 4:3:3.

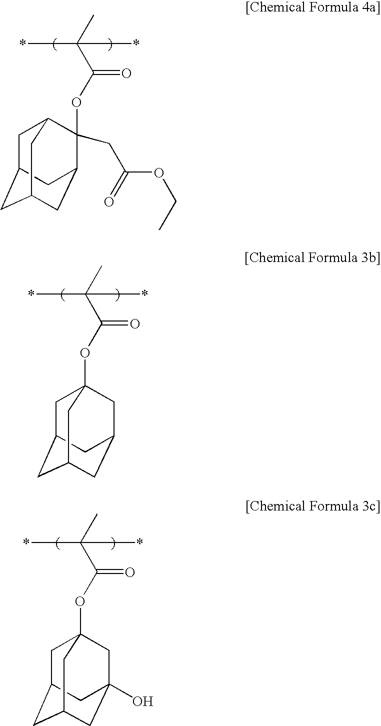

### Example 2-3

### Synthesis of Photosensitive Polymer

40 mmol of the monomer (III) synthesized according to Example 1-3, 30 mmol of 1-adamantyl methacrylate (AMA), and 30 mmol of 3-hydroxy-1-adamantyl methacrylate (HAMA) were put in a round flask, and then, polymerized according to the same method as in Example 2-1, thus obtaining a photosensitive polymer including repeating units represented by the following Chemical Formulae 5a, 3b, and 3c (yield: 60%). The photosensitive polymer had a weight average molecular weight (Mw) of 7,800 and polydispersity (Mw/Mn) of 1.6. The mole ratio (l:m:n) of the repeating units represented by Chemical Formulae 5a, 3b and 3c was 4:3:3.

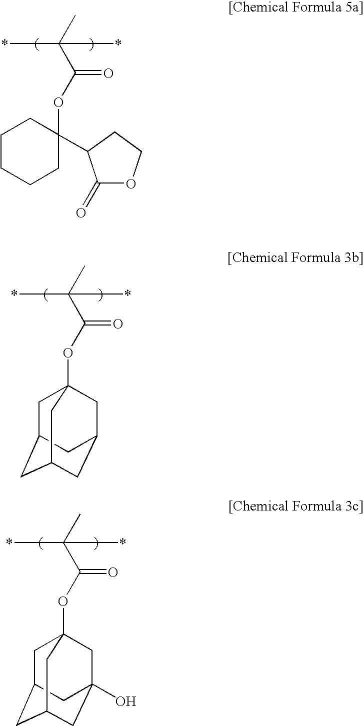

### Example 2-4

### Synthesis of Photosensitive Polymer

40 mmol of the monomer (III) synthesized according to Example 1-3, 30 mmol of 2-ethyl-2-adamantyl methacrylate (EAMA), and 30 mmol of 3-hydroxy-1-adamantyl methacrylate (HAMA) were put in a round, and then, polymerized according to the same method as in Example 2-1, thus obtaining a photosensitive polymer including repeating units represented by the following Chemical Formulae 5a, 6b, and 3c (yield: 60%). The photosensitive polymer had a weight average molecular weight (Mw) of 7,800 and polydispersity (Mw/Mn) of 1.6. The mole ratio (l:m:n) of the repeating units represented by Chemical Formulae 5a, 6b, and 3c was 4:3:3.

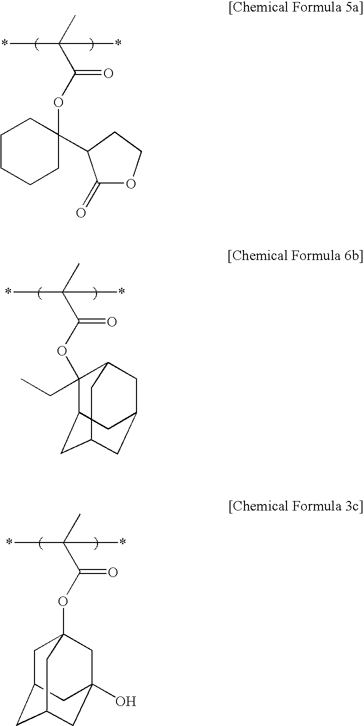

### Example 2-5

### Synthesis of Photosensitive Polymer

40 mmol of the monomer (I) synthesized according to Example 1-1, 30 mmol of 2-methyl-2-adamantyl methacrylate (MAMA), and 30 mmol of 3-hydroxy-1-adamantyl methacrylate (HAMA) were put in a round flask, and then, polymerized according to the same method as in Example 2-1, thus obtaining a photosensitive polymer including repeating units represented by the following Chemical Formulae 3a, 7b, and 3c (yield: 60%). The photosensitive polymer had a weight average molecular weight (Mw) of 8,000 and polydispersity (Mw/Mn) of 1.6. The mole ratio (l:m:n) of the repeating units represented by Chemical Formulae 3a, 7b, and 3c was 4:3:3.

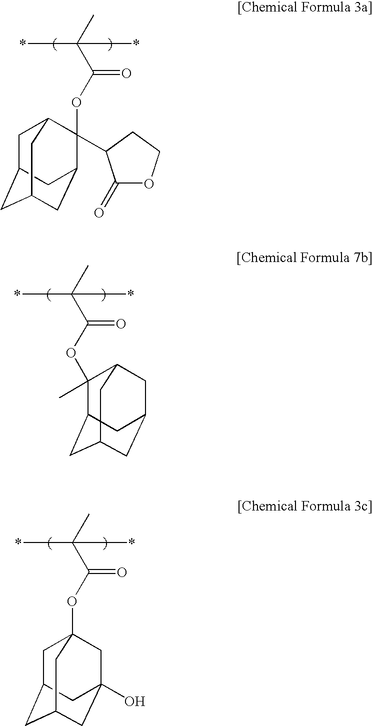

### Example 2-6

### Synthesis of Photosensitive Polymer

40 mmol of the monomer (II) synthesized according to Example 1-2, 30 mmol of 2-methyl-2-adamantyl methacrylate (MAMA), and 30 mmol of 3-hydroxy-1-adamantyl methacrylate (HAMA) were put in a round flask, and then polymerized according to the same method as in Example 2-1, thus obtaining a photosensitive polymer including repeating units represented by the following Chemical Formulae 5a, 8b, and 3c (yield: 60%). The photosensitive polymer had a weight average molecular weight (Mw) of 8,300 and polydispersity (Mw/Mn) of 1.5. The mole ratio (l:m:n) of the repeating units represented by Chemical Formulae 4a, 7b, and 3c was 4:3:3.

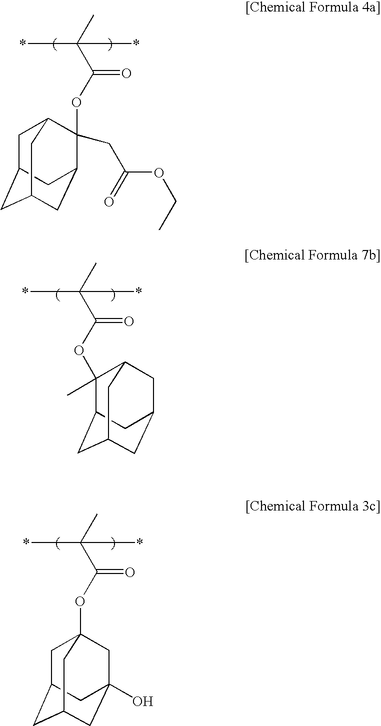

### Example 2-7

### Synthesis of Photosensitive Polymer

40 mmol of the monomer (II) synthesized according to Example 1-2, 30 mmol of 2-ethyl-2-adamantyl methacrylate (EAMA), and 30 mmol of 3-hydroxy-1-adamantyl methacrylate (HAMA) were put in a round flask, and then, polymerized according to the same method as in Example 2-1, thus obtaining a photosensitive polymer including repeating units represented by the following Chemical Formulae 4a, 6b, and 3c (yield: 60%). The photosensitive polymer had a weight average molecular weight (Mw) of 7,800 and polydispersity (Mw/Mn) of 1.5. The mole ratio (l:m:n) of the repeating units represented by Chemical Formulae 4a, 6b, and 3c was 4:3:3.

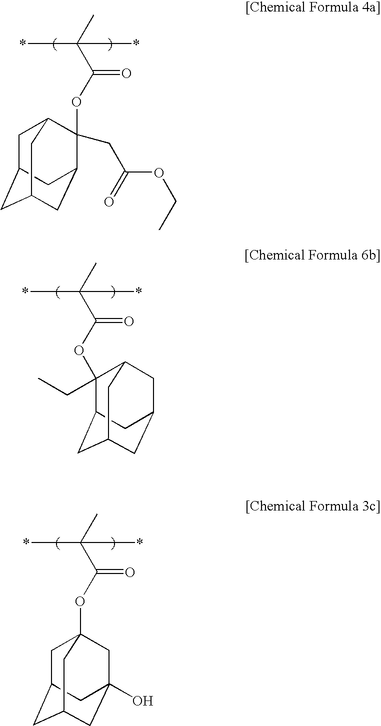

### Example 2-8

### Synthesis of Photosensitive Polymer

30 mmol of the monomer (I) synthesized according to Example 1-1, 30 mmol of 2-methyl-2-adamantyl methacrylate (MAMA), 10 mmol of 1-adamantyl methacrylate (AMA), and 30 mmol of 3-hydroxy-1-adamantyl methacrylate (HAMA) were put in a round flask, and then polymerized according to the same method as in Example 2-1, thus obtaining a photosensitive polymer including repeating units represented by the following Chemical Formulae 3a, 7b, 3b, and 3c (yield: 60%). The photosensitive polymer had a weight average molecular weight (Mw) of 8,100 and polydispersity (Mw/Mn) of 1.6. The mole ratio (l:m:n:k) of the repeating units represented by Chemical Formulae 3a, 7b, 3b, and 3c was 3:2:1:3.

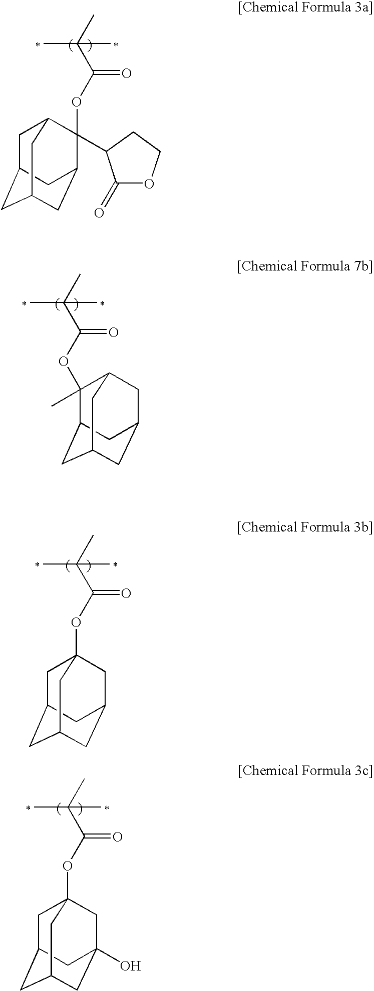

### Example 2-9

### Synthesis of Photosensitive Polymer

30 mmol of the monomer (III) synthesized according to Example 1-3, 20 mmol of 2-ethyl-2-adamantyl methacrylate (MAMA), 10 mmol of 1-adamantyl methacrylate (AMA), and 30 mmol of 3-hydroxy-1-adamantyl methacrylate (HAMA) were put in a round flask, and then, polymerized according to the same method as in Example 2-1, thus obtaining a photosensitive polymer including repeating units represented by the following Chemical Formulae 5a, 6b, 3b, and 3c (yield: 60%). The photosensitive polymer had a weight average molecular weight (Mw) of 8,300 and polydispersity (Mw/Mn) of 1.6. The mole ratio (l:m:n:k) of the repeating units represented by Chemical Formulae 5a, 6b, 3b, and 3c was 3:2:1:3.

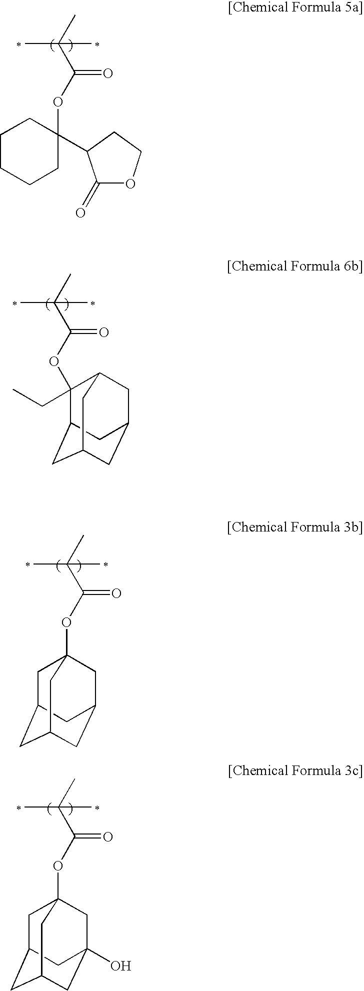

### Examples 3-1 to 3-9

### Preparation of Resist Composition and Lithography Performance

0.8 g of each photosensitive polymer synthesized according to Examples 2-1 to 2-9 was dissolved in 17 g of propylene glycol monomethyl ether acetate (PGMEA)/ethyl lactate (7/3 volume ratio) solvent with triphenyl sulfonium nonaflate photoacid generator. Then, 1 mg of triethanolamine, an organic base, was dissolved in the solution. The resulting solution was filtered by a 0.1 μm membrane filter to prepare resist compositions 3-1 to 3-9.

### Experimental Example 1

### Resolution Evaluation

The prepared resist compositions according to Examples 3-1 to 3-9 were coated to a thickness of 140 nm on a silicon wafer including a 600 Å-thick organic anti-reflecting coating (BARC, AR46, Rhom & Hass Company), and soft-baked (SB) at a temperature of 110° C. for 60 seconds. The coated wafer was exposed to light with an ArF scanner (0.88 NA, annular σ=0.85-0.55), post-exposure baked (PEB), and then developed in a 2.38 wt % tetramethylammonium hydroxide aqueous solution for 60 seconds.

As a result of the developed resist composition prepared according to Example 3-4, a 70 nm line end space pattern was obtained.

The results of developed resist composition prepared according to Examples 3-1 to 3-9 are shown in the following Table 1.

TABLE 1

| | | | | Exposure | |
|---|---|---|---|---|---|
| | | SB | | dose | Resolution |
| Composition | Polymer | (° C.) | PEB (° C.) | (mJ/cm²) | (nm) |
| Example 3-1 | Example 2-1 | 110 | 110 | 35 | 80 |
| Example 3-2 | Example 2-2 | 110 | 110 | 30 | 70 |
| Example 3-3 | Example 2-3 | 110 | 110 | 38 | 70 |
| Example 3-4 | Example 2-4 | 110 | 110 | 30 | 70 |
| Example 3-5 | Example 2-5 | 110 | 110 | 35 | 70 |
| Example 3-6 | Example 2-6 | 110 | 110 | 33 | 70 |
| Example 3-7 | Example 2-7 | 110 | 110 | 35 | 70 |
| Example 3-8 | Example 2-8 | 110 | 110 | 37 | 80 |
| Example 3-9 | Example 2-9 | 110 | 110 | 35 | 70 |

As shown in the Table 1, a clear line end space (L/S) pattern having a resolution of 70 to 80 nm was obtained at a dose of 30 to 38 mJ/cm² in all Examples 3-1 to 3-9.

### Experimental Example 2

### Etching Resistance Evaluation

The photosensitive polymer according to Example 2-1 was evaluated regarding bulk etching according to a RIE (reactive ion etching) method under CF₄ gas (composition: power of 100 W, pressure of 5 Pa, a flow rate of 30 ml/min). As a result of the etching resistance evaluation, the photosensitive polymer according to Example 2-1 exhibited an etching rate of only about 1.10 times an etching rate of a poly(hydroxystyrene) polymer reference, a resist used for KrF. Therefore, it may be seen that the photosensitive polymer synthesized according to Example 2-1 exhibited excellent etching resistance.

The resist material of an embodiment may exhibit excellent dry etching resistance. In other words, the resist material of an embodiment may have sufficient selectivity to avoid difficulties in performing a dry etching process using plasma gas during a semiconductor device manufacturing process.

The resist material of an embodiment may have excellent solubility in a developer solution and adherence to an underlayer as well as excellent dry etching resistance due to e.g., the composition of the photosensitive polymer. In particular, the cyclic groups of the (meth)acrylate compound may enhance dry etching resistance and the acid-labile ester group may enhance adherence to an underlayer, due to, e.g., the non-hydrophobic characteristics of the ester group.

In addition, the resist composition of an embodiment may be formed with sufficient synthesis yield during the preparation of the polymer, and may be prepared at a low cost. Furthermore, the resist composition may exhibit excellent storage-stability.

The resist material of an embodiment may control resist contrast due to, e.g., the acid-labile ester group that may undergo a decomposition reaction under an acid catalyst. Therefore, the resist composition may be usefully applied to fabricate a next generation semiconductor.

Exemplary embodiments have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

What is claimed is:

1. A (meth)acrylate compound having an acid-labile ester group, the (meth)acrylate compound being represented by the following Chemical Formula 1 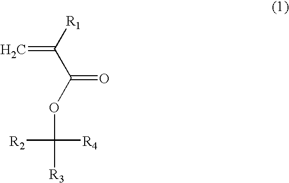
wherein, R₁ is hydrogen or methyl,
R₂ and R₃ are each independently a substituted or unsubstituted linear alkyl, a substituted or unsubstituted cyclic alkyl, or linked to each other to form a cyclic ring or a fused-ring, and
R₄ is a linear ester or cyclic ester group.

2. The (meth)acrylate compound as claimed in claim 1, wherein R₂ and R₃ are the cyclic ring linking R₂ and R₃, the cyclic ring including cyclopentyl, cyclohexyl, adamantyl, or isobornyl.

3. The (meth)acrylate compound as claimed in claim 1, wherein R₄ is γ-butyrolactonyl or —(CH₂)ₙCOOR′, n being an integer of 1 to 3 and R′ being a C1 to C3 alkyl.

4. The (meth)acrylate compound as claimed in claim 1, wherein the (meth)acrylate compound includes at least one compound selected from the group consisting of compounds represented by the following Chemical Formulae 1a to 1j:

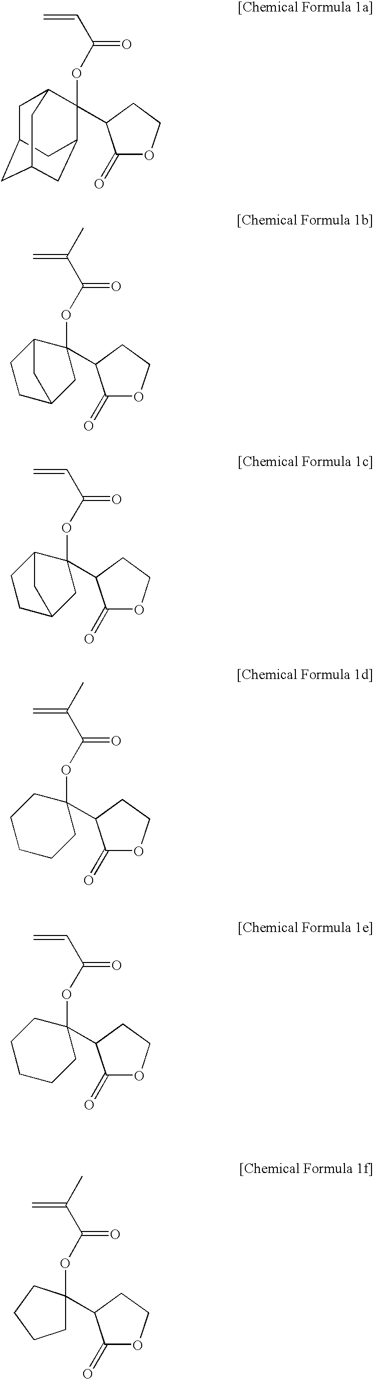

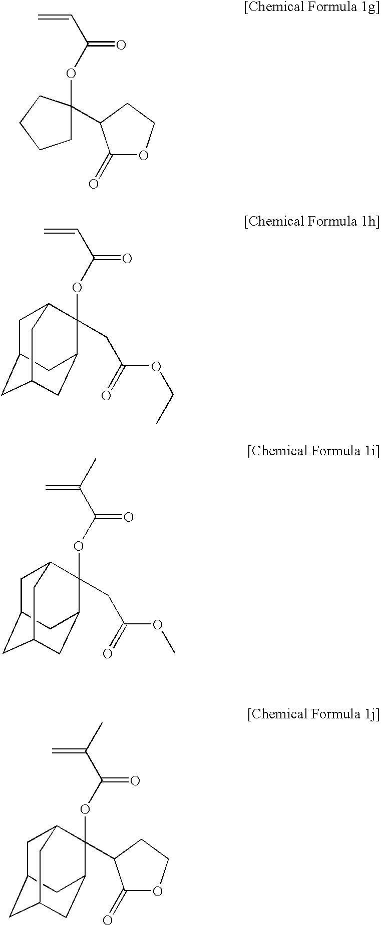

5. A photosensitive polymer, comprising a repeating unit derived from a compound represented by the following Chemical Formula 1: 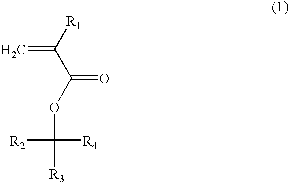
wherein, R₁ is hydrogen or methyl,
R₂ and R₃ are each independently a substituted or unsubstituted linear alkyl, a substituted or unsubstituted cyclic alkyl, or linked each other to form a monocyclic ring or a fused-ring, and
R₄ is a linear ester or cyclic ester group.

6. The photosensitive polymer as claimed in claim 5, wherein the photosensitive polymer has a weight average molecular weight (Mw) of about 3,000 to about 20,000.

7. The photosensitive polymer as claimed in claim 5, wherein the photosensitive polymer has a polydispersity (Mw/Mn) of about 1.3 to about 2.5.

8. The photosensitive polymer as claimed in claim 5, wherein the photosensitive polymer further comprises at least one repeating unit derived from a compound represented by the following Chemical Formula 2: 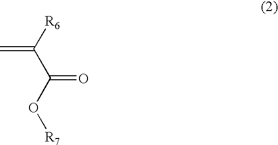
wherein, in Chemical Formula 2, R₆ is hydrogen or methyl, and
R₇ is hydrogen, a bulky alkyl, an alkyl including a polar functional group, or a cycloalkyl including a polar functional group.

9. The photosensitive polymer as claimed in claim 8, wherein R₇ is the bulky alkyl, the bulky alkyl being unsubstituted norbornyl, norbornyl substituted with a lower alkyl, unsubstituted isobornyl, isobornyl substituted with a lower alkyl, unsubstituted cyclodecanyl, cyclodecanyl substituted with a lower alkyl, unsubstituted adamantyl, adamantyl substituted with a lower alkyl, alkoxycarbonyl, alkoxycarbonylalkyl, amyloxycarbonyl, amyloxycarbonylalkyl, 2-tetrahydropyranyloxycarbonylalkyl, 2-tetrahydrofuranyloxycarbonylalkyl, a tertiary alkyl, or acetal.

10. The photosensitive polymer as claimed in claim 8, wherein R₇ is 2-hydroxyethyl or 3-hydroxy-1-adamantyl.

11. The photosensitive polymer as claimed in claim 8, wherein a mole ratio of repeating units derived from compounds represented by Chemical Formula 1 to all repeating units derived from compounds represented by Chemical Formula 2 is about 3:7 to about 7:3.

12. A resist composition, comprising
the photosensitive polymer as claimed in claim 5;
a photoacid generator; and
an organic solvent.

13. The resist composition as claimed in claim 12, wherein the photosensitive polymer is included in an amount of about 5 to about 15 parts by weight, based on 100 parts by weight of the resist composition.

14. The resist composition as claimed in claim 12, wherein the photoacid generator is included in an amount of about 1 to about 15 parts by weight, based on 100 parts by weight of the photosensitive polymer.

15. The resist composition as claimed in claim 12, wherein the photoacid generator includes at least one of triarylsulfonium salts, diaryliodonium salts, and sulfonates.

16. The resist composition as claimed in claim 12, wherein the composition further comprises about 0.1 to about 1.0 part by weight of an organic base, based on 100 parts by weight of the photosensitive polymer.

17. The resist composition as claimed in claim 16, wherein the organic base includes at least one of triethylamine, triisobutylamine, trioctylamine, triisodecylamine, and triethanolamine.
